(19) United States
(12) Patent Application Publication
(10) Pub. No.: US 2006/0259125 A1
(43) Pub. Date: Nov. 16, 2006

(54) System and method for forming composite stent-graft assembly in-situ

(76) Inventor: James C. Peacock III, San Carlos, CA (US)

Correspondence Address: JOHN P. O'BANION;O'BANION & RITCHEY LLP, 400 CAPITOL MALL SUITE 1550, SACRAMENTO, CA, 95814, US

(21) Appl. No.: 11/408,806

(22) Filed: Apr. 21, 2006

Related U.S. Application Data
(63) Continuation of application No. PCT/US04/35534, filed on Oct. 25, 2004.
(60) Provisional application No. 60/514,199, filed on Oct. 23, 2003.

(51) Int. Cl. A61F 2/84; A61F 2/90

(52) U.S. Cl. 623/1.12; 623/1.13

(57) ABSTRACT

A stent-graft system includes a graft member and separate coupling stent that are adapted to be delivered separately to a location within a patient's body where they are coupled to form a stent-graft composite in-situ. The system thus allows for serial introduction of the graft member and coupling stent through an introducer sheath providing access to the location, instead of being delivered as the composite assembly together, thus allowing substantially reduced size of the introducer sheath. Particular embodiments provide highly beneficial improvements for treating AAAs, allowing for Seldinger puncture access techniques versus the conventional highly invasive “cut down” access procedures required by conventional pre-formed AAA stent-graft composite systems.

### CROSS-REFERENCE TO RELATED APPLICATIONS

This application claims priority from, and is a 35 U.S.C. §111(a) continuation of, co-pending PCT international application serial number PCT/US2004/035534, filed on Oct. 25, 2004, incorporated herein by reference in its entirety, which designates the U.S., which claims priority from provisional U.S. Provisional Patent Application Ser. No. 60/514,199, filed on Oct. 23, 2003 and that is co-owned herewith, and which is herein incorporated in its entirety by reference thereto.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. §1.14.

### BACKGROUND OF THE INVENTION

1. Field of the Invention

This invention relates to the field of medical devices, and more particularly to systems and methods for treating aneurysms in the body, and still more particularly for treating abdominal aortic aneurysms.

2. Description of Related Art

Abdominal aortic aneurysms (“AAA”) are a significant medical problem that often may lead to death if left untreated and in the event of rupture. Substantial efforts have been expended to provide therapies for this condition. One series of therapies are direct surgery. Another series of therapies include percutaneous translumenal delivery of endo-aortic stent grafts to the region of the AAA to isolate the compromised aneurysmic wall from harmful endo-aortic blood pressures as an inside-out approach.

The direct surgical efforts to treat aortic aneurysms are major medical undertakings, and are correlated with substantial patient morbidity, long times in the OR, high costs, and still high incidence of ongoing problems. The percutaneous translumenal endo-aortic grafting measures involve substantially large implants within the aorta as the most major artery of the body. They also relate to high patient morbidity associated with surgical “cut-downs” required to gain access into peripheral arteries leading to the aorta, e.g. in the femoral arteries below the bifurcation in the legs. In particular, the tremendous size of the stent-grafts themselves, even when “folded” during delivery and prior to expansion within a AAA, are of substantial size requiring large guide or introducer sheaths. Thus, a “Seldinger” technique of vascular access and transvascular delivery is not generally possible due to these size considerations, though such would be substantially more desirable with lower morbidity to such cut-downs.

According to the substantial shortcomings of existing procedures, both surgical and percutaneous, many early incidences of AAA are left untreated, as such solutions are medically considered more problematic than the problem. Watchful waiting as the AAA progresses becomes the lifestyle of such patients that would otherwise be considered lucky for catching a AAA early before it becomes potentially deadly. Once the AAA progresses to critical dilation, only then is one of the conventional invasive procedures undertaken.

There is still a need for a lower profile solution to delivering and deploying stent-grafts within aneurysms in order to treat the aneurysms, and in particular with respect to the substantially large thoracic and abdominal aortic aneurysms.

There is also still a need for improved MA and thoracic aortic stent-graft systems and methods providing percutaneous translumenal therapy to the aneurysms via less-invasive Seldinger puncture access techniques.

There is also still a need for improved systems and methods for treating AAAs with reduced patient morbidity.

A need also still exists for a stent-graft approach to treat aortic aneurysms that is appropriately safe and efficacious to allow treatment of aneurysms at early diagnosis and before they reach the later, more dangerous advanced stages of dilation.

### BRIEF SUMMARY OF THE INVENTION

The invention therefore provides various aspects that are considered generally beneficial over prior efforts to treat aortic aneurysms, and in particular thoracic aortic aneurysms and AAAs. In general, where various aspects of the present invention are described for AAAs, further aspects also contemplate similar beneficial improvements as applied or modified appropriately for thoracic aortic aneurysm therapy as well.

The invention according to one aspect provides a percutaneous translumenal solution to treating AAA's via a Seldinger wound puncture technique for vascular access.

Another aspect of the invention is a system and method that provides acceptable therapy for early diagnosed AAAs.

Another aspect of the invention is a system and method that provides reduced profile stent-graft system for treating AAAs.

Another aspect of the invention is a system and method that provides medically acceptable prophylaxis of AAA progression in early diagnosed AAAs.

Another aspect of the invention is a system and method for treating AAAs with improved patient morbidity versus prior direct surgical and percutaneous translumenal AAA therapies that require surgical “cut-downs” for vascular access.

Another aspect of the invention is an AAA stent-graft system and method that is adapted to provide less-invasive therapy to AAAs via less-invasive Seldinger puncture access techniques and percutaneous translumenal delivery for implantation within the AAA.

Another aspect of the invention is a system and method for treating AAAs that provides the stent and graft assemblies separately through a AAA introducer sheath, and that are adapted to couple with each other within the body and distally from the introducer sheath lumen to thereby provide lower profile delivery, and thus lower profile introducer sheaths, and thus adapted to form a stent-graft composite assembly in-situ.

Another aspect of the invention is a stent-graft system that includes a modular stent-graft assembly comprising a stent and a graft member. The stent and graft member are adapted to be separately delivered to a location within a patient's body. Also included in the system is means for combining the stent and the graft member to form a composite stent-graft assembly in-situ at the location. The in-situ formed composite stent-graft assembly is adapted to be implanted at a location within the patient's body.

Another aspect of the invention is a stent-graft system that includes a modular stent-graft assembly comprising a stent and a graft member as follows. The stent and graft member are adapted to be separately delivered to a location within a patient's body. The stent and graft member are adapted to be combined to form a composite stent-graft assembly in-situ at the location. Also included in the system is a means for implanting the in-situ formed composite stent-graft assembly within the patient's body.

Another aspect of the invention is a stent-graft system that includes a modular stent-graft assembly comprising a stent and a graft member as follows. A means for separately delivering the stent and graft member to a location within a patient's body is provided. The stent and graft member are adapted to be combined to form a composite stent-graft assembly in-situ at the location. The in-situ formed composite stent-graft assembly is adapted to be implanted within the patient.

Another aspect of the invention is a stent-graft system that includes a modular stent-graft assembly comprising a graft member and a stent as follows. The graft member and stent are adapted to be separately delivered to a location within a body of a patient. The graft member and stent are adapted to be combined to form a composite stent-graft assembly in-situ at the location. The in-situ formed composite stent-graft assembly is adapted to be implanted at an implant location within the patient.

Another aspect of the invention is a stent-graft system that includes a graft member that is adapted to be delivered to a location within a patient's body. Also included is a graft coupler assembly provided along the graft member. The graft coupler assembly is adapted to couple to and engage a mating stent coupler assembly from a coupling stent to form a composite stent-graft assembly in-situ at the location. The in-situ formed composite stent-graft assembly is adapted to be implanted at an implant location within the patient.

According to one mode of this aspect, the system further includes a stent with a stent coupling assembly. The stent and graft member together comprise a modular stent-graft assembly as follows. The stent and graft member are adapted to be delivered separately to the location. The stent and graft member are adapted to be combined via coupling of the stent coupling assembly and the graft coupling assembly so as to form a composite stent-graft assembly in-situ at the location. The in-situ formed composite stent-graft assembly is adapted to be implanted at the location.

Another aspect of the invention is a stent-graft system that includes a stent that is adapted to be delivered to a location within a patient's body. A stent coupler assembly is provided along the stent. The stent coupler assembly is adapted to couple to and engage a mating graft coupler assembly of a graft member so as to form a composite stent-graft assembly in-situ at the location. The in-situ formed composite stent-graft assembly is adapted to be implanted at an implant location within the patient.

According to one mode of this aspect, the system further comprises a graft member with a graft coupling assembly. The stent and graft member together comprises a modular stent-graft assembly as follows. The stent and graft member are adapted to be delivered separately to the location. The stent and graft member are adapted to be combined via coupling of the stent coupling assembly and the graft coupling assembly so as to form a composite stent-graft assembly in-situ at the location. The in-situ formed composite stent-graft assembly is adapted to be implanted at the location.

Various further modes and embodiments are contemplated that provide further particular benefit in furtherance of the other various aspects noted above.

In particular such mode, a stent-graft system according to one or more of the aspects and modes above further includes an introducer sheath with an introducer lumen. The stent and graft member are each adapted to be separately delivered to the location through the introducer lumen of the introducer sheath.

According to one embodiment of this mode, the introducer sheath is a femoral access introducer sheath. In another related embodiment, the introducer sheath is adapted to provide peripheral vascular access using a Seldinger technique. In another embodiment, the introducer lumen comprises an inner diameter and the in-situ formed composite stent-graft assembly comprises an outer profile that is larger than the inner diameter of the introducer lumen.

According to another embodiment a delivery member is provided with a delivery lumen is provided as follows. The delivery member is adapted to be delivered to the location through the introducer lumen. The stent and graft members are adapted to be delivered to the location through the delivery lumen.

According to another modular composite stent-graft assembly mode, each of the stent and graft member is adapted to be separately delivered to a location within the patient's vasculature in a radially collapsed condition. The in-situ formed composite stent-graft assembly comprises a radially collapsed condition; whereas the in-situ formed composite stent-graft assembly is adapted to be expanded from the radially collapsed condition to a radially expanded condition at the implant location.

In another modular stent-graft assembly mode, the in-situ formed composite stent-graft assembly is adapted to be delivered from the location to a separate implant location.

In another modular stent-graft assembly mode, the stent and graft member are adapted to be combined to form the composite stent-graft assembly in-situ at the location that is substantially positioned at the implant location.

According to still another mode of one or more of the foregoing aspects or modes providing a stent for modular in-situ formation of a stent-graft composite, the stent comprises a self-expanding stent.

In one embodiment of this mode, the self-expanding stent comprises a network of struts constructed from a superelastic alloy material. In a further embodiment, the superelastic alloy material comprises a nickel-titanium alloy.

According to another mode, a stent coupler assembly provided in the system includes a plurality of stent couplers located at spaced intervals around a circumference of the stent. Each of the stent couplers is adapted to couple with a unique one of a plurality of graft couplers of a graft coupling assembly and that are provided at spaced intervals around a circumference of the graft member.

According to one embodiment of this mode, each stent coupler includes a guiderail tracking member that is adapted to slideably engage and track over a guiderail engaged with a corresponding graft coupler such that the stent coupler is registered with the corresponding graft coupler in-situ.

According to another mode providing a modular graft for in-situ combination with a stent, the graft member comprises a substantially pliable, substantially tubular wall. In one embodiment of this mode, the graft member comprises a fluoropolymer. In another embodiment, the graft member comprises polytetrafluoroethylene (PTFE). In still another embodiment, the graft coupler assembly comprises a plurality of graft couplers located at spaced intervals around a circumference of the substantially tubular wall. Each graft coupler is adapted to couple with a unique one of a plurality of stent couplers of a stent coupling assembly provided along a stent. In one particular, highly beneficial further variation of this embodiment, each of the graft couplers is adapted to cooperate with one of a plurality of guiderails, such that each of the graft couplers is adapted to register with each of a plurality of stent couplers tracked over the respective guiderails for in-situ coupling therebetween the stent and graft couplers.

According to another modular stent-graft composite assembly mode, a plurality of guiderails are provided in the system as follows. The graft coupler assembly comprises a plurality of graft couplers that are each adapted to engage a separate one of the guiderails. The stent coupler assembly includes a plurality of stent couplers that each comprises a guiderail tracking member that is adapted to slideably engage and track over one of the guiderails so as to register with one of the graft couplers for in-situ coupling therewith at the location.

In one embodiment of this mode, each of the guiderails is detachably engaged with a respective one of the graft couplers. In a further embodiment, each of the guiderails is electrolytically detachably engaged with a respective one of the graft couplers. In a still further embodiment, each of the guiderails includes an eletrolytically sacrificial joint. In another still further embodiment, an electrical power source is provided that is adapted to be electrically coupled to the electrolytically sacrificial joint such that a circuit may be created sufficient to electrolytically dissolve the sacrificial joint.

According to another modular stent-graft composite assembly mode, each of the stent and graft member comprises an outer profile size that is less than about 18 F. The in-situ formed composite stent-graft assembly is adapted to be implanted in a radially expanded condition at an implant location along a AAA as a AAA stent-graft composite assembly. In one embodiment of this mode, the in-situ formed composite stent-graft assembly in the radially expanded condition comprises an expanded diameter of between about 20 millimeters and about 36 millimeters. Further to this embodiment regarding outer expanded diameters, in additional modes, each of the stent and graft member may include a still smaller outer profile size for delivery that is less than about 14F, and in other modes is less than about 12 F, and in still further modes is less than about 10 F.

According to another modular stent-graft assembly mode, the in-situ formed stent-graft assembly is adapted to be implanted at an implant location along a AAA.

In another mode, the in-situ formed stent-graft assembly is adapted to be implanted at an implant location along a thoracic aortic aneurysm.

In still another mode, the system further includes an anchor assembly that is adapted to anchor the in-situ formed composite stent-graft assembly to a tissue wall at the implant location.

Another aspect of the invention is a method for treating a vascular condition that includes delivering a graft member to a location within a patient's body, delivering a stent to the location separately from the graft member, coupling the stent to the graft member to form a composite stent-graft assembly in-situ at the location, and implanting the in-situ formed composite stent-graft assembly at an implant location within the patient's body.

According to one mode of this aspect, the graft member and stent are separately delivered to the location in series through an introducer sheath.

According to another mode, the stent is coupled to the graft member to form the composite stent-graft assembly in-situ at the location by coupling a plurality of stent couplers associated with the stent with a plurality of graft couplers associated with the graft member.

According to one embodiment of this mode, the method further includes delivering the graft member to the location before delivering the stent to the location, and guiding each of the plurality of stent couplers to each of the plurality of graft couplers at the location over a plurality of guiderails extending proximally from the graft couplers at the location and externally of the patient.

In another embodiment, the method further includes delivering the stent to the location before delivering the graft member to the location, and guiding each of the plurality of graft couplers to each of the plurality of stent couplers at the location over a plurality of guiderails extending proximally from the stent couplers at the location and externally of the patient.

In another mode of the present method aspect of the invention, the method further includes implanting the in-situ formed composite stent-graft assembly at the implant location by releasing the stent from a retainer assembly and allowing the stent to self-expand. Further to this mode, the composite stent-graft assembly expands to engage a vessel wall at the implant location under force of expansion from the self-expanding stent.

In another mode, the in-situ formed composite stent-graft assembly is implanted along a AAA.

In still another mode, the in-situ formed composite stent-graft assembly is implanted along a thoracic aortic aneurysm.

In still another mode, the method also includes anchoring the in-situ formed composite stent graft assembly at the implant location.

In yet another mode, the method also includes combining the stent and graft assembly in-situ at the location to form the composite stent-graft assembly in a radially collapsed condition, and expanding the in-situ formed composite stent-graft assembly from the radially collapsed condition to a radially expanded condition at the implant location.

Another aspect of the invention includes a stent delivery system as follows. A self-expanding stent is provided with a plurality of networked interconnected struts and that is adjustable between a radially expanded configuration that is a shape memory condition for the stent and a radially collapsed configuration that is an elastically deformed condition for the stent under an applied radial retention force away from the shape memory condition. A delivery member is also provided with a plurality of longitudinal tethers spaced about a circumference around the delivery member and each having a length between distal and proximal ends and being threaded in a radially undulating pattern that alternates between valleys that are coupled to the delivery member and peaks that extend radially away from the delivery member and over a multiple longitudinally spaced segments of the interconnected strut network of the stent. Each of the plurality of longitudinal tethers is adjustable between a first condition that is held taught to retain the stent in the radially collapsed condition and a second condition that is longitudinally released and proximally withdrawn from the stent to thereby release the stent for expansion to the radially expanded configuration.

Each of the foregoing aspects, modes, and embodiments is considered independently beneficial without requiring further combination with the others or other components or elements, notwithstanding whether such benefit may be provided for example simply by providing the ability to ultimately provide such combination. Notwithstanding the foregoing, the various combinations apparent to one of ordinary skill are further beneficial and independent aspects contemplated hereunder.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawing which is for illustrative purposes only:

FIG. 1 shows a AAA treatment procedure during a first mode of operation providing access to an AAA via a Seldinger puncture access technique with an AAA introducer sheath according to one embodiment of the invention.

FIG. 2 shows the delivery of a graft member of a separate stent-graft system through the AM introducer sheath and to a location within the AAA according to a further mode of the embodiment shown in FIG. 1.

FIG. 3 shows the delivery of a coupling stent into an interior passageway of the graft member at the location where they are coupled to form a stent-graft assembly in situ within the AAA according to a further mode of the embodiment.

FIG. 4 shows the in-situ formed stent-graft assembly in an expanded condition following coupling of the coupling stent with the graft member and is the implanted condition for the stent-graft assembly adapted to substantially isolate and protect the AM wall from interior abdominal aortic blood flow within the interior passageway along the implanted stent-graft assembly.

FIG. 5 shows a transverse cross-section through the distal end of one graft member adapted for use in the system variously shown in FIGS. 1-4 according to a further embodiment of the invention, and shows cross-sections of certain folding stylets adapted to assist in folding the graft member into a relatively low profile delivery configuration that is further adapted to provide an interior passageway with pre-arranged positions for graft couplers to allow advancement of a coupling stent through the passageway and locking engagement between the graft couplers and corresponding stent couplers on the coupling stent.

FIG. 6 shows a transversely cross-sectioned view of a graft member similar to that shown in FIG. 5 but in a further mode that is wound or folded in a manner that is adapted for low profile, modular Seldinger delivery and in-situ coupling with a stent within a AAA.

FIG. 7 shows a partially transversely cross-sectioned angular perspective view of a graft member in a folded configuration such as shown in FIG. 6 within an AM introducer sheath.

FIG. 8 shows a side view of a schematic representation of certain detail of a coupling stent and related stent delivery system adapted for delivery and coupling within a graft member such as shown in FIGS. 5-6, and according to the overall stent-graft system variously shown in FIGS. 1-4.

FIG. 9 shows a partially transversely cross-sectioned angular perspective view of a coupling stent, such as similar to that shown in FIG. 7, after delivery within an interior passageway of a folded graft member, such as similar to that shown in FIG. 6, and after certain stent couplers of the coupling stent are coupled in locked engagement within corresponding graft couplers of the graft member, and after withdrawal of the stent delivery system, and thus shows a collapsed configuration for the in-situ formed stent-graft assembly, such as according to the mode of use shown in FIG. 3.

FIG. 10 shows a transversely cross-sectioned view through a distal end of the in-situ formed stent-graft assembly of FIG. 9, except shown in an expanded configuration adapted as an implant within a AAA, such as according to the mode of use shown in FIG. 4.

FIG. 11 shows a schematic transversely cross-sectioned view of another graft member during one mode of folding operation according to another embodiment adapted to provide a shaped interior passageway for slideable advancement of a coupling stent.

FIG. 12 shows a schematic transversely cross-sectioned view of the graft member shown in FIG. 11 in a folded configuration within an AAA introducer sheath.

FIG. 13 shows a longitudinally cross-sectioned side-view of a coupling stent during advancement over electrolytically detachable guiderails within an interior passageway of a graft member that provide a registered coupling between stent couplers that track over the rails and graft couplers that provide annular housings through which the guiderails extend and that are adapted to receive the stent couplers sliding over the guiderails in a friction fit, and further shows the guiderails secured to the graft member tip at securement points through holes formed into and between two adjacent laminated walls as a cuff formed by an inverted tip portion of the graft member.

FIG. 14 shows an exploded angular perspective view of a tip portion of a guiderail that is adapted to be fixed within the securement points, with a flattened portion having an aperture allowing for bonding of opposite walls of the inverted tip portion of the graft member for improved securement, and also showing a reduced diameter portion of the guiderail as an electrically conductive wire that is of such dimension to provide for electrolytic dissolution of the joint for detachment of the round wire portion from the securement point following stent-graft coupling.

FIG. 15 shows a schematic transversely cross-sectioned view of a four-coupler configuration for a graft member with four wing folds that is also adapted for use according to the overall system shown schematically in various modes of operation in FIGS. 1-4.

### DETAILED DESCRIPTION OF THE INVENTION

It is to be appreciated according to the various aspects of the invention described above, and by reference to the various FIGS. and accompanying brief descriptions, that various aspects of the present invention provide for the separate percutaneous translumenal delivery of a graft member and separate coupling stent into an AAA, where they are coupled in-situ to form a stent-graft assembly. This arrangement allows for reduction of overall profile during delivery, and each of the two coupling components are delivered within the same introducer sheath, but not at the same time. Instead, they are delivered therethrough in series, e.g. with the graft first and then the coupling stent. Thus, their individual profiles are less than the profile that would result if they were both coupled radially together as a composite. Accordingly, introducer sheaths of dramatically reduced interior lumen diameters, and thus profiles, may be used versus conventional AAA stent-graft assemblies. In particular, it is believed that according to the present invention profile reductions may be achieved sufficient to allow Seldinger puncture access techniques to be used.

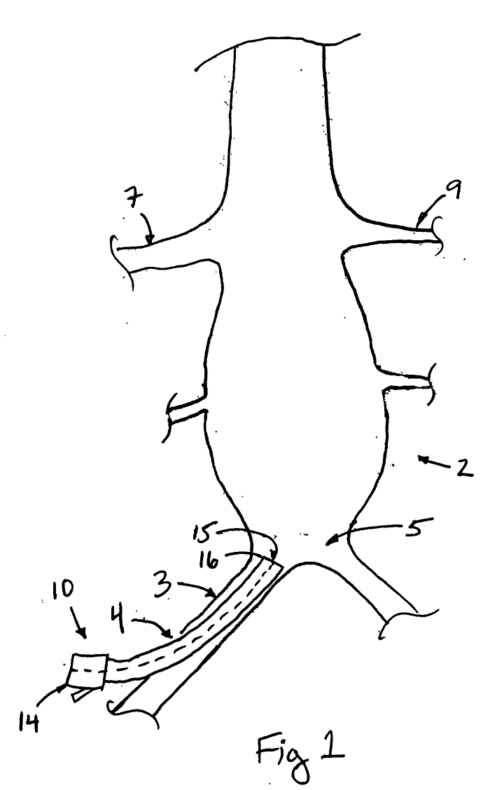

Turning now to the particular illustrative embodiments, FIG. 1 shows a AAA treatment procedure during a first mode of operation providing percutaneous translumenal access to an AAA 2 via a Seldinger puncture access site 4 along a femoral artery 3 and using a related technique with an AAA introducer sheath 10. More specifically, AAA introducer sheath 10 includes an elongate body 12 with an introducer lumen 15 (shown in shadow) that extends between a proximal coupler 14 and a distal tip 16 where an end port is provided. Proximal coupler 14 may be for example a hemostatic valve, etc., or otherwise be fitted for coupling to such a valved coupler to provide for hemostasis when devices are introduced therethrough lumen 15 and into the abdominal aorta. Introducer sheath 10 is in particular adapted of appropriate dimension and material construction to allow for relatively atraumatic retrograde delivery of devices from the access site 4 along femoral artery 3 and beyond the femoral bifurcation 5 into AAA 2. According to various aspects of the present invention, such introducer device and related Seldinger technique is sufficient to allow for delivery of a AAA stent graft assembly without requiring substantial surgical cut-down of the femoral artery 3.

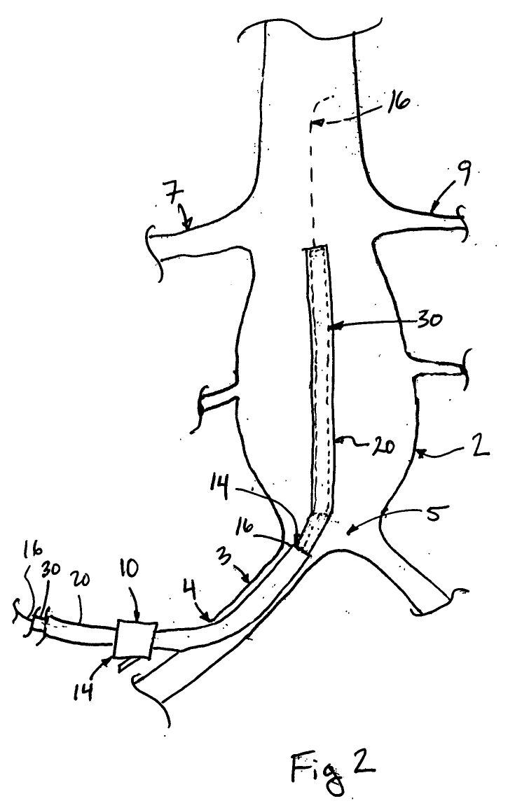

FIG. 2 shows the procedure initiated as shown in FIG. 1, but in a further sequential mode. More specifically, a further delivery sheath 20 is shown extended distally from distal tip 16 of introducer sheath 10 and retrogradedly along AAA 2 to an infrarenal location below renal arteries 7,9. Such is accomplished for example over a guidewire 16 (shown in shadow). Shown schematically within delivery sheath 20 is a graft member 30 of a modular stent-graft system that will be explained in further detail by reference to certain particular illustrative embodiments below.

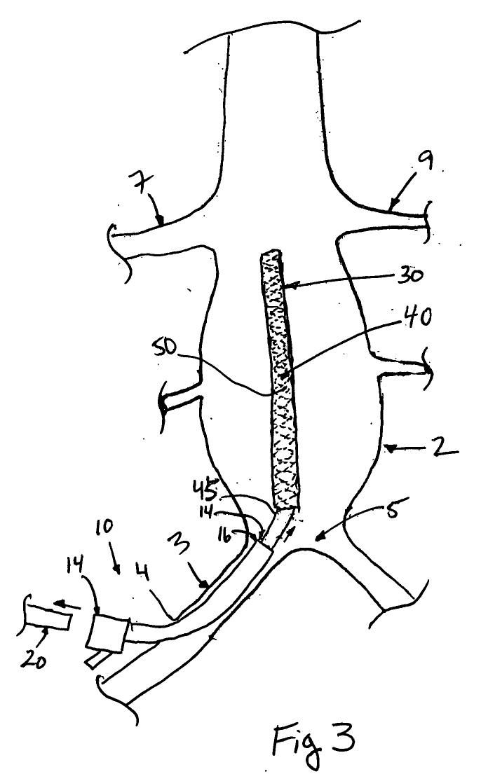

FIG. 3 shows still a further sequential mode of operation according to the procedure initiated and developed by reference to FIGS. 1-2 above. More specifically, delivery sheath 20 shown in FIG. 2 has been withdrawn. By such withdrawal of external sheath 20, graft member 30 is left exposed within AAA 2 such that it may be expanded there as an implant once coupled to an expansion mechanism, such as a self-expanding stent according to further embodiments hereunder.

Also shown in FIG. 3 is a coupling stent assembly 40 that includes a coupling stent 50 releasably engaged with a stent delivery member 45 that delivers the stent 50 through introducer sheath 10 sequentially after graft member 30 is introduced therethrough. Stent delivery member delivers stent 50 distally from introducer sheath 10 and into an interior passageway defined by a folded tubular wall of graft member 30 within AAA 2.

In this arrangement, graft member 30 coaxially surrounds coupling stent 50 within AAA 2, though they were separately delivered through introducer sheath 10. As will be further developed below by reference to certain particular illustrative further embodiments, a mechanism is included within the combined modular stent-graft assembly which provides for pre-determined and controlled positioning relationship and coupling between stent 50 and graft member 30 in this coaxial arrangement. In particular embodiments, for example, one or more guiderails are coupled to certain locking mechanisms or couplers at predetermined positions along graft member 30, and over which stent 50 is tracked for engaged coupling. By delivering coupling stent 50 into the interior passageway of the graft member 30 at the location of AAA 2 in this manner, they are thus coupled to form a stent-graft assembly 60 in situ within AAA 2 in a position where the composite is to be implanted.

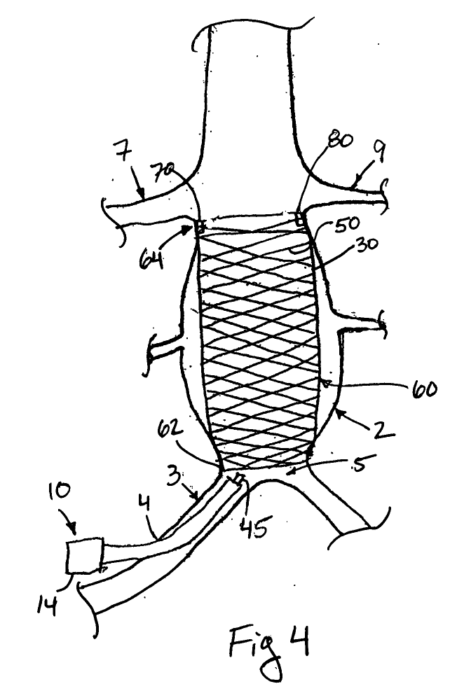

FIG. 4 shows yet a further sequential mode according to the procedure described above by reference to FIGS. 1-3. Here, the in-situ formed stent-graft assembly 60 is shown in an expanded condition following coupling of the coupling stent 50 with the graft member 30. This is the implanted condition for the stent-graft assembly 60 and is adapted to substantially isolate and protect the wall of AAA 2 from interior abdominal aortic blood flow within the interior passageway along the implanted stent-graft assembly. As shown in FIG. 4, stent 50 is released from stent delivery member 45, which may be withdrawn from the body through introducer sheath 10. Stent graft composite assembly 60 extends between a proximal end portion 62 and a distal end portion 64 and spans the length of AAA 2 to the extent necessary to provide the necessary isolation from the blood pool, shown in FIG. 4 in one exemplary illustration to extend from a relatively high infra-renal position to a relatively low position distally adjacent to the femoral bifurcation.

For general understanding and illustration of the overall in-situ stent-graft coupling scheme, exemplary coupling members 70,80 are schematically shown in FIG. 4 at distal end 64 of stent graft composite assembly 60 within graft member 30, and will be explained in further detail below.

Various modes and mechanisms may be employed to provide the in-situ stent-graft coupling described for the various embodiments. Certain particular embodiments are described as follows for the purpose of providing further detail of certain illustrative approaches to achieve this objective.

According to the embodiment shown in partial transverse cross-section in FIG. 5, the distal end 94 of one illustrative graft member 90 is shown in one mode of use during folding in preparation for delivery to a AAA 2 for in-situ stent coupling and implantation. More specifically, graft member 90 includes a tubular wall 92 that is folded relatively flat to form two opposite folds 96,98. Shown within an annular passageway 93 of tubular wall 92 are cross-sectioned portions of coupling members 100,110. Coupling members 100,110 are located opposite each other relative to a second transverse axis T2 that is transverse to first transverse axis T1. In the particular embodiment shown, coupling members 100,110 provide annular seats that are slideably engaged with first and second guide rail members 106,116 along the interior of graft member 90. Further detail of this assembly and its function in operation is provided below.

In any event, from this initially flat folded configuration, a number of different methods and tools may be employed to roll or fold graft member 90 into a substantially tight, low-profile configuration for delivery through a Seldinger introducer sheath and into a AAA 2. In the particular beneficial embodiment shown, a plurality of folding stylets 99 are provided and positioned in a manner that is adapted to assist in folding the graft member into a relatively low profile delivery configuration that is further adapted to provide an interior passageway with pre-arranged positions for graft couplers to allow advancement of a coupling stent through the passageway and locking engagement between the graft couplers and corresponding stent couplers on the coupling stent.

Further to the particular arrangement shown in FIG. 5, graft couplers 100,110 are positioned so as to remain adjacent each other in a collapsed central passageway formed despite folding or rolling of folds 96,98 around this central region. This is possible in this arrangement, even though the couplers 100,110 are located in relatively fixed locations 180 degrees apart from each other around the circumference of graft member 30. According to this relative positioning of the graft couplers 100,110, a stent may be delivered through the respective central region of a folded assembly for efficient coupling between the stent and the graft at locations that are separated 180 degrees apart. This will provide substantial benefit to further intended operation, and in particular to assist in unitary expansion of the in-situ coupled composite, and as further developed below.

For further illustration of the present embodiment, FIG. 6 shows one folded form of the graft member 30 shown in FIG. 5 in the flat configuration immediately prior to forming this folded configuration. In the folded configuration of FIG. 6, each of two folds 96,98 are wrapped like folded wings around the central region where couplers 100,110 are located. In this particular embodiment, the wings are each wrapped in a clockwise fashion around a central region that serves as a stent passageway 91 where opposite couplers 100,110 are located in adjacent confronting orientations.

Stylets 99 are shown in FIG. 6 in their respective working positions to assist in the folding process, acting as supports around which the folds 96,98 are wrapped. In addition, these stylets 99 may remain in place during delivery to the AAA, providing structural support to the otherwise flaccid graft member 90. While four stylets 99 are shown here at 90 degree intervals around couplers 100,110, it is to be appreciated that different combinations, numbers, sizes, or locations of stylets may be employed as helpful tools.

Stylets 99 may be required to provide axial support over a substantial length, e.g. over the length of graft member 90, in particular to assist in the folding. However, if used also for support during in-situ delivery, then a certain degree of flexibility may be required, especially where relatively tortuous femoral passage is required to the AAA. Still further, as typical graft member materials such as PTFE are not generally radiopaque, stylets 99 (in embodiments using them for in-vivo delivery support) may also include a radiopaque material of construction, such as for example similar to conventional guidewire constructions with substantially strong core wire members wrapped by more radiopaque but softer coil members. In one particular beneficial embodiment, stylets 99 are metal mandrels, such as for example but without limitation stainless steel, cobalt-chromium, or a superelastic or shape memory alloy such as nickel-titanium alloy. Again, in any case a simple wire core construction may be sufficient for folding purposes, or for graft delivery purposes. But, where radiopacity is desired for such stylets, additional radiopaque additives, materials, or members for construction may be employed.

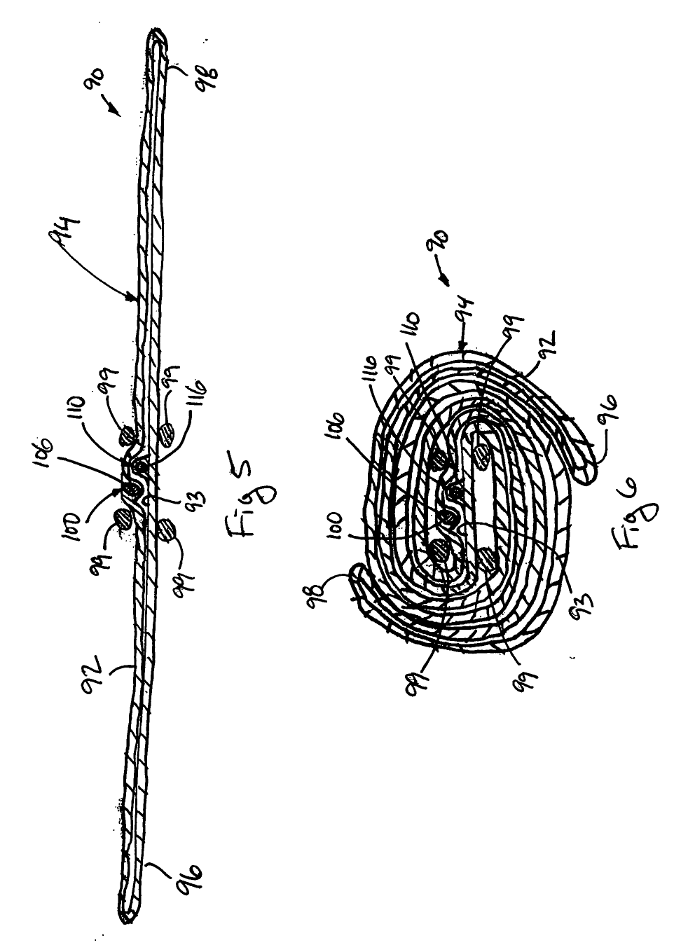

The folded configuration for graft member 90 shown in FIG. 6 is adapted for low profile slideable engagement within a delivery passageway of a delivery member. This is illustrated in FIG. 7 that shows a partially transversely cross-sectioned angular perspective view of graft member 90 shown in the same folded configuration as FIG. 6, but after positioning within an introducer passageway 122 of an introducer sheath 120.

As shown in FIG. 7, folded graft member 90 is housed within introducer passageway 122 with just the right clearance to allow for slideable passage therethrough with an optimally low profile system. Because there is no stent involved in the graft delivery according to the present embodiment of the invention, this profile may be substantially reduced than if graft member 90 were coupled also to a stent at this stage during use for introduction and delivery to an AAA. In other words, if the stent component were pre-coupled to the graft prior to introduction in a more conventional arrangement, the stent would add appreciable size to a collapsed stent-graft composite versus just providing the graft member as shown in FIG. 7. That added size from the stent would require a much larger introducer lumen for passage, which would require a larger outer profile for the introducer sheath, ultimately requiring a substantially larger entry wound into the patient. Accordingly, the present invention substantially benefits over prior conventional stent-graft approaches that deliver the composite together in unitary form and according to much higher profiles during delivery.

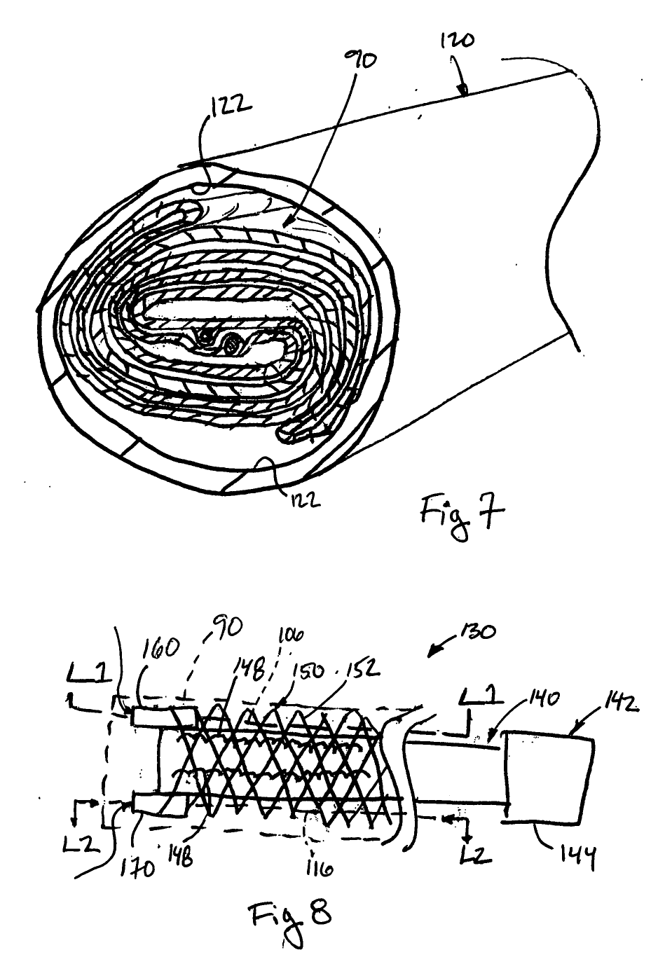

It is to be appreciated that FIGS. 5, 6, and 7 illustrate sequential modes or providing a graft member 90 for delivery to a AAA for in-situ combination with a stent within the AAA. FIGS. 8-9 provide certain further detail related to the stent delivery aspect of the present embodiments as follows.

FIG. 8 shows certain detail, though in generally schematic form, of one coupling stent assembly 130 adapted for use with a graft member 90 and other related components of the system variously described by reference to FIGS. 1-7. More specifically, coupling stent assembly 130 includes a stent delivery assembly 140 coupled to a coupling stent 150. Stent delivery assembly 140 includes a proximal assembly 142 with an actuator 144, and a delivery member 145 that includes a plurality of stent retainers 148. Coupling stent 150 includes a stent 152 to which guiderail couplers 160,170 are secured. Further details of construction and related to the interactive operation of stent delivery assembly 140, coupling stent 150, and graft member 90 are described by various reference to FIGS. 8 and 9 as follows.

Coupling stent 150 is typically constructed as a self-expanding type, such as of an interconnected network of struts constructed of a superelastic alloy material such as nickel titanium alloy. The memory condition for the stent 150 is in the expanded configuration, whereas it is shown in FIG. 8 in a collapsed formed condition for delivery. Stent 150 is held in the superelastically deformed, collapsed condition by means of stent retainers 148 that are threads that are threaded radially over and around struts of stent 150 and tightened down onto delivery member 145. For example, such threads may be threaded in serpentine manner along the length of delivery member 145, alternately extending along a lumen within delivery member (not shown), and externally of that lumen and around struts of stent 150, such as via a linear array of ports through the thread lumen. An external view of this is shown schematically in FIG. 8. In any event, a circumferential array of such longitudinal threads is provided that together hold stent 150 collapsed onto delivery member 145 for delivery.

Guiderail couplers 160,170 are tubular members with lumens 166,176 and are arranged to slideably engage and track over guiderails 106,116 engaged with graft member 90 along a longitudinal axis, e.g. shown for illustration at respective axes L1 and L2 in FIG. 8. This is done, for example, by backloading proximal end portions of guiderails 106,116 extending externally of the patient into and through the guiderail couplers 160,170 when the guiderails extend proximally from graft member 90 positioned within AAA 2.

In the collapsed configuration of stent assembly 150 shown in FIG. 8, a certain clearance is provided within stent 150 to allow for passage of guiderails 106,116 within the confines of the stent 150 proximally of guiderail couplers 160,170. However, this is one illustrative embodiment, and other arrangements may be provided though not herein specifically shown in detail. For example, guiderail couplers 160,170 may extend the length of stent 150, either within the tubular wall of networked struts, or exteriorly thereof. Or, the guiderails may be more integrated into the network of stent struts, such that for example the tubes are spines along the stent 150 between which the lattice network of the stents circumferentially extend, such as for example by welding strut ends of a lattice patterned sheet directly to a series of circumferentially spaced longitudinal hypotubes. Any arrangement achieving the objectives set forth herein or otherwise apparent to one of ordinary skill is contemplated within the broad intended scope of the present invention.

FIG. 9 shows a partially transversely cross-sectioned angular perspective view of coupling stent 150 previously shown in FIG. 8, but after delivery within interior stent passageway 91 of folded graft member 90 within AAA 2. The arrangement shown is after guiderail couplers 160,170 of the coupling stent 150 are coupled in locked engagement within corresponding graft couplers 100,110 of the graft member 90, and the stent delivery system 140 is removed from the picture for clarity. In any event, the coupled combination is achieved as follows.

As shown in FIG. 9, graft couplers 100,110 are provided in the form of annular seats. These seats are adapted to receive the guiderail couplers 160,170 of stent assembly 150 in seated engagement therein, such as for example in a friction fit. This is achieved for example by tracking the guiderail couplers 160,170 into the annular seats over the guiderails (not shown for clarity of other features) as they extend through those seats 100,110 in the engagement mode of use for graft member 90. Seats or graft couplers 100,110 thus may be for example of a material and or geometry to allow for the required passage of guiderails therethrough and to coaxially receive the guiderail couplers, but such that the guiderail couplers once received therein become engaged in a substantially secure manner to allow for unitary manipulation to an expanded implant condition as a composite. For example, as mentioned above, this may be via a friction fit, or otherwise in a keyed fitting, detent lock, ratchet lock, or other suitable engagement.

Once guiderail couplers 160,170 are seated and engaged within couplers 100,110, stent 150 is released from delivery member 145 by releasing the retainers 148 (e.g. see FIG. 8), such as for example by withdrawing them through their sewn arrangement between stent 150 and delivery member 145. Such adjustment may include for example electrolytic detachment of metallic or otherwise conductive threads from a distal attachment point. Or, the threads may be simple frangible upon sufficient proximal tension force to release or break from a distal attachment and thus allow withdrawal.

In still another regard, a thread may be in the form of a loop that extends both along the undulating sewn longitudinal axis as shown schematically in FIG. 8, but also loops back and out from the patient at both ends. In this way, it may be held taut at both ends (and thus the sewn looped region between) for delivery purposes and the collapsed configuration for the stent 150, and then one end could be pulled with the other end released, threading the released end back through the catheter and past the sewn portion with the stent 150.

In any event, upon release of the retainers 148, stent 150 is allowed to self-expand. However, the particular arrangement of FIG. 9 is shown immediately before self-expansion of the stent and after withdrawal of the stent delivery system for clarity of illustration, though likely the self-expansion is achieved fairly rapidly upon release of stent retainers 148 and before actual withdrawal of stent delivery system 140 (by reference to FIGS. 8 and 9). Thus, stent 150 is still shown in FIG. 9 in a collapsed configuration for the in-situ formed stent-graft assembly 160, such as according to the mode of use shown schematically in FIG. 3. Further to this arrangement though, it is to be appreciated that the overall outer profile of the composite with the stent 150 engaged within the graft member 90 is larger than either the folded configuration for the graft member 90 alone, as shown in FIG. 6, or for the delivery collapsed configuration of stent 150. In fact, if the two components were provided pre-coupled in a composite before introduction, the inner diameter of the introducer sheath would be required to house the assembly shown in FIG. 9, rather than the smaller introducer capable of introducing these components as serial parts (e.g. FIG. 7).

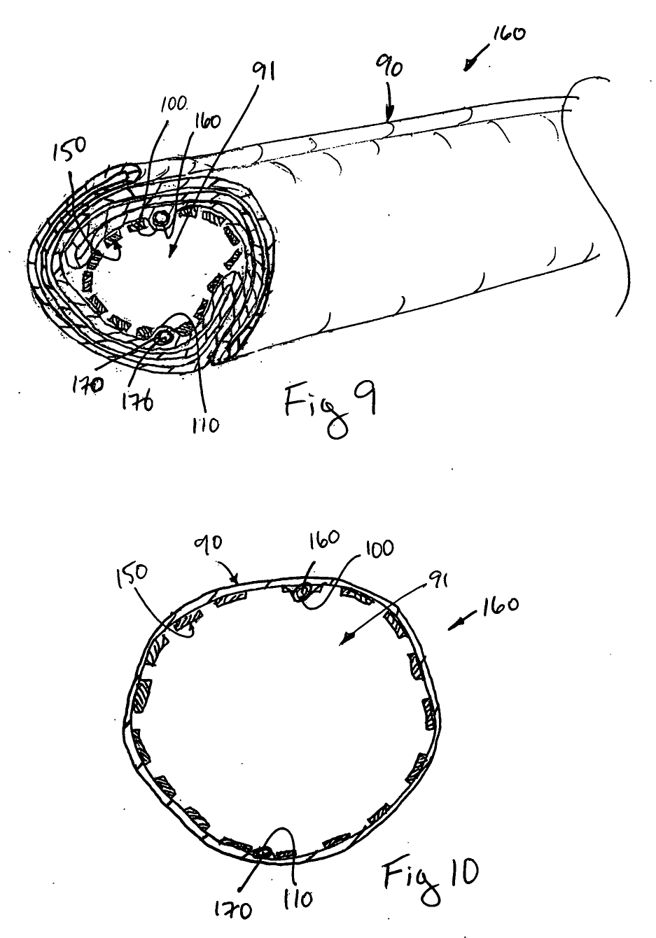

Composite stent graft assembly 160 is shown in FIG. 10 after release and self-expansion of stent 150 in the coupled configuration with graft member 90. Further shown schematically is cross-sectioned stent-strut segments which, per the self-expanding memory recovery, push open the graft member 90 around its circumferencial wall until resistance is encountered at the aorta wall (or until the material of the stent is completely recovered to its memory shape). In general though, a stent-graft is chosen of appropriate size such that the diameter of the wall to be engaged is equal to or slightly less than the recovery diameter of the stent. As such, a kit of appropriately matched stents and graft members is provided, with a range of sizes to accommodate a range of patient anatomies. For the sake of providing completely clear illustration of the interrelationships between the various components of the overall system herein described, FIG. 10 shows the cross section taken so that the position of the graft couplers 100,110 are depicted in spacial relationship according to this particular embodiment.

However, it is to be appreciated that the particular features of the foregoing embodiment, and according to the various aspects expanded upon variously through FIGS. 1-10, may be modified or improved upon without departing from the intended scope hereof. Moreover, various particular details of one or more features not heretofore described may also provide particular benefits and are contemplated hereunder.

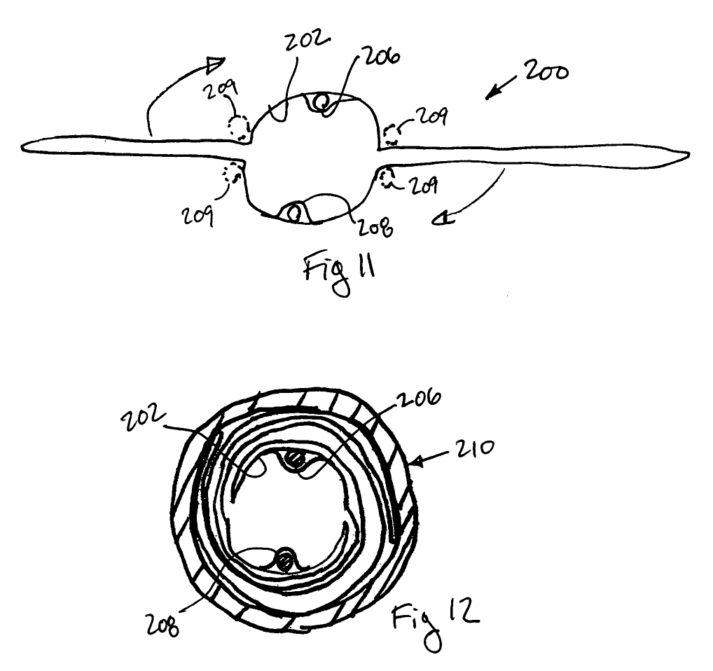

In one particular regard, FIG. 11 shows a schematic cross-section of a slightly different initial fold configuration for a graft member 200 similar to that shown in FIG. 5, but in a manner providing more real estate for the stent passageway 202 allowing for coaxial delivery and coupling of a delivered stent with the coupling members 206,208 provided. Stylets 209 may be provided in a similar manner and for similar purposes as described above with respect to the embodiment of FIG. 5. The result of this slightly modified fold configuration with expanded stent passageway 202 is shown within an introducer sheath 210 in FIG. 12, whereas in still a further variation the stent passageway 202, though available to this geometry and diameter for housing a stent for coupling, may be collapsed under radial compression forces during delivery through the introducer sheath 210.

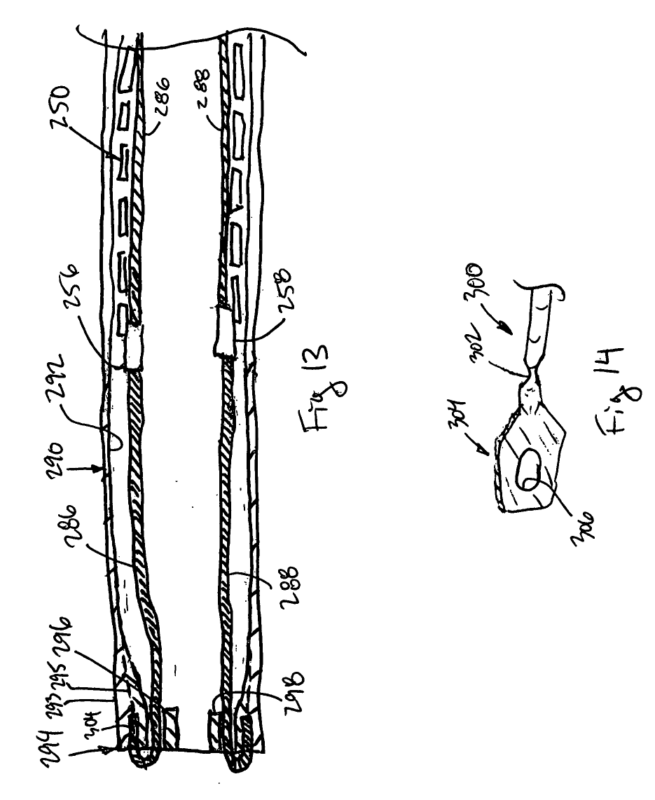

It is also to be appreciated that various particular modes of construction and operation are contemplated that suitably provide for stent delivery separately from the graft member and for in-situ coupling of the stent with the graft member. One particular further embodiment of this aspect is shown in FIG. 13 in longitudinal cross-section, and is intended to be read in conjunction and context with the description above and in particular relation to FIG. 8.

More specifically, FIG. 13 shows a longitudinally cross-sectioned side-view of a coupling stent 250 during advancement over electrolytically detachable guiderails 286,288 within an interior passageway 292 of a graft member 290. The guiderails 286,288 provide a registered coupling between guiderail couplers 256,258 that track over the rails 286,288 and graft couplers 296,298, respectively. Graft couplers 296,298 provide annular housings through which the guiderails 286,288 extend, and are adapted to receive the stent couplers 256,258, respectively, sliding over the guiderails 286,288 such that stent couplers 256,258 are held in a friction fit within graft couplers 296,298.

As further shown in FIG. 13, the guiderails 286,288 are secured to the graft member tip 294 at securement points between two laminated walls 293,295, such as may be formed as a cuff by an inverted or everted edge portion of a wall of the graft member 290 that is secured to itself following inversion (or eversion). Such may be accomplished for example either by heatbonding or welding, adhesive bonding, or other technique as would be apparent to one of ordinary skill.

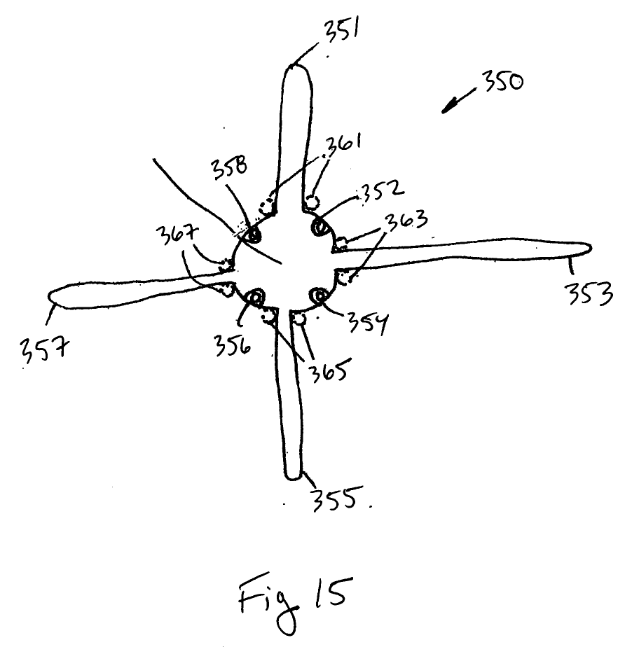

Further detail of certain features related to the guiderail securement and detachment to graft member 250 is shown in FIG. 14, and described by reference thereto and further reference to FIG. 13. More specifically, electrolytic detachment joints 282,284 are shown along guiderails 286,288, respectively, at locations distal to graft couplers 296,298 and adjacent to their respective securement points to the graft wall. This is shown in further detail in FIG. 14, which is a necked or otherwise thinned portion of the metallic and conductive members forming the rails. Also shown in FIG. 14 is a further beneficial feature in one illustrative example, providing a flattened tip portion 304 of an exemplary guiderail 300 and located distally adjacent the respective detachment joint 302 of that guiderail 300. Through this flattened tip portion 304 is an aperture or pore 306. When flattened tip 304 is positioned between the adjacent wall portions of graft member 290 to form a securement there, e.g. by melting the inverted wall portions together around flattened tip portion, the pore 306 allows communication therethrough. This greatly enhances the bond and securement of tip portion 304 in the tip of graft member 290.

According to the foregoing, the securement of flattened tip portion 304 of each guiderail into graft member 290 is sufficiently robust to prevent dislodging during tracking of guiderail couplers 160,170 over the guiderails 106,116 and into graft couplers 100,110. Thereafter once the guiderail couplers 160,170 and graft couplers 100,110 are coupled to each other, an electrical current is applied to the respective guiderails in a manner which electrolytically dissolves the detachment joints, such as shown at illustrative joint 302. Such detachment and related equipment and techniques and methods may be similar for example to that provided for electrolytically detachable embolic coils. Furthermore, this electrolytic detachment approach, and related systems, materials, and equipment, may be furthermore related to highly beneficial modes of releasing the retension members from the stent delivery member 145.

Electrolytic detachment is noted in various embodiments and related features, and generally may employ modified and new applications of known assemblies and methods for achieving the present embodiments and related objects. This may include for example the systems, devices, materials, and methods previously used for delivering and detaching embolic coils for aneurysm treatments, e.g. the Guglielmi Detachable Coil (GDC) commercially available by Boston Scientific.

The disclosures of the following issued US Patents, and in particular without limitation to the extent providing more detailed examples of electrically dissolved medical device implant detachment systems and methods as variously disclosed in one or more of these references, are herein incorporated in their entirety by reference thereto: U.S. Pat. No. 5,851,206 to Guglielmi et al.; U.S. Pat. No. 5,855,578 to Guglielmi et al.; U.S. Pat. No. 5,895,385 to Guglielmi et al.; U.S. Pat. No. 5,919,187 to Guglielmi et al.; U.S. Pat. No. 5,925,037 to Guglielmi et al.; U.S. Pat. No. 5,928,226 to Guglielmi et al.; U.S. Pat. No. 5,944,714 to Guglielmi et al.; U.S. Pat. No. 5,947,962 to Guglielmi et al.; U.S. Pat. No. 5,947,963 to Guglielmi; U.S. Pat. No. 5,976,126 to Guglielmi; U.S. Pat. No. 5,984,929 to Bashiri et al.; U.S. Pat. No. 6,010,498 to Guglielmi; U.S. Pat. No. 6,015,424 to Rosenbluth et al.; U.S. Pat. No. 6,066,133 to Guglielmi et al.; U.S. Pat. No. 6,086,577 to Ken et al.; U.S. Pat. No. 6,156,061 to Wallace et al.; U.S. Pat. No. 6,165,178 to Bashiri et al.; U.S. Pat. No. 6,193,708 to Ken et al.; U.S. Pat. No. 6,375,669 to Rosenbluth et al.; U.S. Pat. No. 6,425,893 to Guglielmi; U.S. Pat. No. 6,425,914 to Wallace et al.; U.S. Pat. No. 6,468,266 to Bashiri et al.; U.S. Pat. No. 6,658,288 to Hayashi; and U.S. Pat. No. 6,716,238 to Elliott. The disclosures of these references are herein incorporated in their entirety by reference thereto.

While such electrolytic detachment feature is considered highly beneficial, other mechanisms may also be used to achieve the objectives of the subject technology according to the present embodiments and related broad aspects. Several other devices and techniques may be used to provide for fixed engagement of components during one mode of operation, followed by detatchment thereof, and are contemplated as further aspects, modes, and/or embodiments of the present invention.

Among other various embodiments that are also herein contemplated, the specific fold configuration of the graft member component of the modular in-situ formed composite may take many forms and shapes other than previously described above. In particular, other configurations or variations of the previously disclosed clockwise, double wing wrapped geometry referenced above and shown in preceding figures may be employed. In addition, various numbers and relative positioning of graft member couplers, and related stent couplers for in-situ coupling therebetween, are also contemplated.

In one particular further example to these points, FIG. 15 shows another embodiment where a graft member 350 includes four folds 351,353,355,357 at 90 degree intervals about a circumference. Also provided are four graft couplers 352,354,356,358, also located at 90 degree circumferential intervals, but at inward invaginated portions between adjacent graft wall folds. Here, four pairs of stylets 361,363,365,367 are also provided on opposite sides of each fold to assist in folding, and/or support assistance during delivery of graft member 350, as similarly described for other embodiments above. However, similar to the other embodiments fewer stylets may be used (e.g. one per fold), or none may be required, depending upon the particular situation.

Particular embodiments shown that are adapted to achieve this purpose are considered highly beneficial, but are considered illustrative of broad aspects of the invention and in that regard are not intended to be limiting.

In further embodiments not shown, the coupling stent may be delivered first, and thereafter the graft member delivered over the stent. In such case, the locations and arrangements of couplers and other components may be modified. For example, the stent couplers may be located along a radially outer periphery of the stent struts, and the guiderails would be engaged with the stent couplers (vs. the graft couplers in the previous embodiments), so as to provide for proper advancement of the graft member relative to the first-positioned stent, and to guide the graft couplers to the stent couplers. Moreover, the male-female couplers and resulting friction fit coupling shown for example in the prior embodiments may be interchanged between the stent and graft couplers in this modification.

Other couplers than those shown may be used to provide in-situ locking engagement with the graft member and coupling stent. Ratchet lock mechanisms may be used, detents, key-in-lock arrangements, or other modes to achieve a friction fit. Moreover, other techniques such as in-situ adhesive bonding may be employed, such as for example by delivering a two-part or UV-cured adhesive into the area of controlled contact or engagement between the stent and coupler, including accompanying delivery lumens or devices as appropriate. Such may be used instead of, or in combination of the coupler mechanisms herein shown and described, and other combinations or modifications thereof are contemplated to the extent consistent with the broad aspects described.

Moreover, in-situ coupling may be achieved by other mechanisms than pre-arranged couplers that are fixed to the respective graft member or coupling stent. Other modes may include for example a remotely operable sewing assembly that attaches the stent and graft together within the AAA by external control outside the body. Such suturing techniques may employ commercially available tools in a new application for this objective and in this way, or may modify such tools to the extent desired to achieve the present objectives and as would be apparent according to a review of this disclosure.

Various materials and assembly techniques may be used for the various components herein described, as would be apparent to one of ordinary skill based upon a review of this disclosure. In many cases,

The various aspects, modes, and embodiments of the invention are herein generally described by reference to providing improved therapies to AAAs, and inparticular by providing a substantial benefit via reduced delivery profiles when compared to other prior efforts. In one regard, such specific dimensions contemplated may relate to the particular application of the present invention in improving upon other design features provided by other conventional composite stent graft systems.

For example, the following are generally believed to be the profiles of various previously disclosed AAA stent-graft composite systems: about 24 F (“Talent” device, commercially available from Medtronic, Inc.), (“Ancure” device, commercially available from Guidant Corporation, and believed to have been the first commercially approved device in the World market); about 22 F (“Aneurex” device commercially available from Medtronic, Inc., and believed to have been the first commercially approved device in the United States market); about 14 F TriVascular/Boston. Another previously disclosed AAA stent-graft composite assembly under the name “Endologics” (C. R. Bard) is believed to be a 28 F profile composite system. Other disclosed AAA composite stent-graft device systems include: “Excluder” device from W. L. Gore; “Zenith” device from Cook Inc.; “Corvita” device from Boston Scientific; “LifePass” device from Baxter; “Quantum” device from Johnson & Johnson Corp.; “Anaconda” device from Sulzer; and “Vanguard” from Boston Scientific.

Accordingly, the range of these device profiles is generally between about 22-28 F, with more recently disclosed yet unapproved devices intended to provide about as low as 14 F for delivery. These profile parameters for delivery are generally a related result to the composite structures necessary to achieve the end result of the implanted device, which may be typically between for example about 20 mm and about 36 mm in expanded diameter of the composite stent-graft assembly.

According to application of one or more of the various embodiments of the present invention, the profile of each of these other approaches may be improved upon by at least about 25%, and in many cases up to even about 50% by providing a modular approach for stent and graft delivery for in-situ combination within the body. In one particular regard, various highly beneficial applications of the present embodiments are capable of providing profile sizes for delivery of less than about 18 F, and still further less than about 14 F, and in still further highly beneficial embodiments equal to or even less than about 12 F. Moreover, even lower maximum outer profile measurements, e.g. less than about 10 F, are believed to be achievable by providing the stent and graft members separately for in-situ combination within the body. In other words, according to the present invention the stent and graft members separately define the maximum introduction or delivery profile to be experienced, whereas their combined composite form is not experienced during delivery through an introducer. Variously at these much reduced delivery profiles, it is contemplated that similar expansion profiles may be achieved when compared to the other conventional pre-formed composite structures. Accordingly, the ratio of delivery profile to expansion profile is also significantly improved, improving thus on the efficiency of the overall system.

Similar relationships may be achieved with other applications of the present embodiments than specifically AAA stent-graft assemblies. For example, stent-graft limb sizes (e.g. for side-branch grafting at the femoral bifurcation in conjunction with a MA stent-graft) may range for example between about 16 F to about 18 F, whereas about 16-18 mm expansion diameters are a typical range for such modular stent-graft branches. Similar ratio of improvement to these profiles is also believed achievable according to the present invention.

It is in particular highly beneficial to provide such lower profiles to the extent allowing for a Seldinger technique to be used for vascular access, as elsewhere herein noted. However, it is to be appreciated that even if surgical cut-downs were to be used according to use of the present embodiments, reduced profiles even in that context are still beneficial goals. Moreover, even to the extent other techniques may be developed that make Seldinger techniques possible for MA stent-grafting for example, still the present embodiments are considered to still provide various improvements thereover (e.g. by still further reducing the profiles below the Seldinger “threshold”, or by achieving the objectives in a better way).

The present embodiments are highly beneficial for use in treating AAAs. However, the various devices, components, and related methods, may be used in other applications, such as to treat other forms or locations of aneurysms, either of the vasculature or otherwise. Suitable modifications may be made in order to achieve the particular objectives of such a specific application without departing from the broad intended scope hereof. For example, thoracic aneurysms elsewhere in the aorta may be treated, and aneurysms of the ascending aorta or aortic arch are in particular deadly conditions that may be treated with such anticipated modifications of the present disclosure.

Other areas where tissue support or isolation is desired from a stent-graft may also be well suited for therapy according to such modified applications of the present disclosure, either related to the cardiovascular system or otherwise. In one particular example, damaged heart tissue, e.g. infarct or congestive heart failure, has been treated, at least in research and development efforts, and in some clinical arenas, with external scaffolding support placed around the heart, e.g. the ventricles for example. A suitably constructed stent member and corresponding graft material may be provided for serial delivery through an introducer assembly, and in-situ coupling between them to form a stent-graft assembly, for the purpose of providing such support scaffold implant, according to further embodiments that are herein contemplated.

In another example, co-pending and co-owned Published International PCT Patent Application Serial No. PCT/US04/34106, entitled “ANEURYSM TREATMENT SYSTEM AND METHOD”, and filed Oct. 14, 2004, is herein incorporated by reference thereto. Among other aspects, this disclosure provides a AAA stent-graft assembly as an external support surrounding the exterior of a AAA (or other location or form of aneurysm). This is generally performed via laparoscopic minimally invasive delivery techniques and related systems, such as for example through intercostals spaces or otherwise along the posterior back of the patient, or in another example via transperitoneal delivery approach and systems from an anterior location. In general, however, this disclosure provides broad and novel minimally invasive delivery systems and methods for AAA and other aneurysm repair systems that improve upon conventional surgical and endovascular approaches in many circumstances.

It is to be appreciated that the systems and methods just described immediately above provide a still further suitable application for modified assemblies and methods of the present disclosure. For example, much benefit is experienced by separately providing the stent and graft components of an external stent-graft support assembly for minimally invasive delivery. Among other benefits, such modular approach allows for smaller introducer devices and respective incisions or “ports” in a similar manner as it allows for smaller introducers, and in many cases makes Seldinger technique of delivery possible, for endovascular applications.

In still a further example, stent-graft assemblies have also been disclosed for providing bypass plumbing from one part of the body to another, such as in particular coronary artery bypass. Further more particular disclosures provide such via minimally invasive or “port” access techniques through smaller incisions than typically used in direct surgical approaches. By providing such stent-grafts modified according to the present disclosure, the size of such introduction devices and incisions may again be reduced substantially.

In addition, the various embodiments herein shown and described generally provide a modular stent-graft system according to particular modes where a graft member is first delivered to an implant location and then a guide system provides for in-situ coupling with a later delivered (i.e. in series) coupling stent. However, further embodiments contemplate suitable modifications of the present detailed embodiments to provide the stent to the location first, followed by a guided registration and coupling with a graft member. In a similar regard, the detailed embodiments and Figures generally provide for illustration a stent located within a graft member to provide the in-situ formed composite. However, still further appropriate modifications may be made according to one of ordinary skill to instead provide the stent along the outer periphery of the graft member in their combined composite form.

The invention has been discussed in terms of certain particular embodiments. One of skill in the art will recognize that various modifications may be made without departing from the scope of the invention. In addition, while particular cooperating or adjunctive treatment or other accessory devices are described for use in conjunction with the present embodiments, other modifications are contemplated as would be apparent to one of ordinary skill. Moreover, while certain features may be shown or discussed in relation to a particular embodiment, such individual features may be used on the various other embodiments of the invention.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

1-3. (canceled)

4. A stent-graft system, comprising:
a modular stent-graft assembly comprising a graft member and a stent;
a graft coupler assembly associated with the graft member;
a stent coupler assembly associated with the stent;
wherein the graft member and stent are configured to be separately delivered to a location within a body of a patient;
wherein the graft member and stent are configured in a manner that is adapted to be combined via a coupling between the graft coupler assembly and the stent coupler assembly so as to form a composite stent-graft assembly in-situ at the location; and
wherein the in-situ formed composite stent-graft assembly is adapted to be implanted at an implant location within the patient.

5-6. (canceled)

7. A method for treating a vascular condition, comprising:
delivering a graft member to a location within a patient's body;
delivering a stent to the location separately from the graft member;
coupling the stent to the graft member by coupling a graft coupler assembly of the graft member with a stent coupler assembly of the stent so as to form a composite stent-graft assembly in-situ at the location; and
implanting the in-situ formed composite stent-graft assembly at an implant location within the patient's body.

8. The system of claim 54, further comprising:
a graft coupler assembly associated with the graft member;
wherein the stent and graft member are adapted to be combined via coupling of the stent coupler assembly and the graft coupler assembly at least in part via the guiderail so as to form a composite stent-graft assembly in-situ at the location.

9. The system of claim 4, wherein:
the stent and graft member are adapted to be combined via coupling of the stent coupler assembly and the graft coupler assembly at least in part via a guiderail so as to form a composite stent-graft assembly in-situ at the location.

10. (canceled)

11. The system of claim 12, wherein the introducer sheath comprises a femoral access introducer sheath.

12. The system of claim 4, further comprising:
an introducer sheath with an introducer lumen;
wherein the introducer sheath is adapted to provide peripheral vascular access using a Seldinger technique.

13. The system of claim 12, wherein:
the introducer lumen comprises an inner diameter; and
the in-situ formed composite stent-graft assembly comprises an outer profile that is larger than the inner diameter of the introducer lumen.

14. The system of claim 4, wherein:
each of the stent and graft member is adapted to be separately delivered to a location within the patient's vasculature in a radially collapsed condition;
the in-situ formed composite stent-graft assembly comprises a radially collapsed condition; and
the in-situ formed composite stent-graft assembly is adapted to be expanded from the radially collapsed condition to a radially expanded condition at the implant location.

15-16. (canceled)

17. The system of claim 4, wherein:
the stent comprises a self-expanding superelastic nickel-titanium alloy stent.

18-19. (canceled)

20. The system of claim 4, wherein:
the stent coupler assembly comprises a plurality of stent couplers located at spaced intervals around a circumference of the stent;
the graft coupler assembly comprises a plurality of graft couplers located at spaced intervals around a circumference of the graft member; and
each of the stent couplers is adapted to couple with a unique one of the graft couplers.

21-26. (canceled)

27. The system of claim 4, further comprising:
a plurality of guiderails;
wherein the graft coupler assembly comprises a plurality of graft couplers that are each coupled to a separate one of the guiderails; and
wherein the stent coupler assembly comprises a plurality of stent couplers that each comprises a guiderail tracking member that is adapted to slideably engage and track over one of the guiderails so as to register with one of the graft couplers for in-situ coupling therewith at the location.

28. The system of claim 27, wherein each of the guiderails is detachably engaged with a respective one of the graft couplers.

29. (canceled)

30. The system of claim 28, wherein each of the guiderails comprises an eletrolytically sacrificial joint.

31. (canceled)

32. The system of claim 12, further comprising:
a delivery member with a delivery lumen;
wherein the delivery member is adapted to be delivered to the location through the introducer lumen; and
wherein the stent and graft members are adapted to be delivered to the location through the delivery lumen.

33-44. (canceled)

45. The method of claim 7, wherein the stent is coupled to the graft member to form the composite stent-graft assembly in-situ at the location by coupling a plurality of stent couplers associated with the stent with a plurality of graft couplers associated with the graft member.

46. The method of claim 45, further comprising:
delivering the graft member to the location before delivering the stent to the location; and
guiding each of the plurality of stent couplers to each of the plurality of graft couplers at the location over a plurality of guiderails extending proximally from the graft couplers at the location and externally of the patient.

47. The method of claim 45, further comprising:
delivering the stent to the location before delivering the graft member to the location; and
guiding each of the plurality of graft couplers to each of the plurality of stent couplers at the location over a plurality of guiderails extending proximally from the stent couplers at the location and externally of the patient.

48. The method of claim 45, further comprising:
implanting the in-situ formed composite stent-graft assembly at the implant location by releasing the stent from a retainer assembly and allowing the stent to self-expand; and
wherein the composite stent-graft assembly expands to engage a vessel wall at the implant location under force of expansion from the self-expanding stent.

49. The method of claim 7, wherein the in-situ formed composite stent-graft assembly is implanted along an abdominal aortic aneurysm (AAA).

50. The method of claim 7, wherein the in-situ formed composite stent-graft assembly is implanted along a thoracic aortic aneurysm.

51. The method of claim 7, further comprising anchoring the in-situ formed composite stent graft assembly at the implant location.

52. The method of claim 7, further comprising:
combining the stent and graft assembly in-situ at the location to form the composite stent-graft assembly in a radially collapsed condition; and
expanding the in-situ formed composite stent-graft assembly from the radially collapsed condition to a radially expanded condition at the implant location.

53. The system of claim 14, comprising:
a delivery member;
a plurality of tethers spaced about a circumference around the delivery member;
wherein each of the plurality of tethers is adjustable between a first condition that is held taught to retain the stent in the radially collapsed condition and a second condition that is released and proximally withdrawn from the stent to thereby release the stent for expansion to the radially expanded configuration.

54. The system of claim 4, further comprising:
a guiderail; and
wherein the graft member and stent are adapted to be combined via a coupling between the graft coupler assembly and the stent coupler assembly at least in part via the guiderail to form a composite stent-graft assembly in-situ at the location.

55. The method of claim 7, further comprising:
coupling the stent and graft member in-situ via at least one guiderail coupled to the stent and graft member, respectively.

56. The method of claim 7, further comprising:
delivering the stent and graft member separately through an introducer lumen of an introducer sheath and to the location via a Seldinger vascular access technique.
